(19) United States
(12) Patent Application Publication
(10) Pub. No.: US 2017/0319085 A1
(43) Pub. Date: Nov. 9, 2017

(54) MULTIFUNCTIONAL HEALTHCARE MONITORING APPARATUS

(71) Applicant: Physio-Control, Inc., Redmond, WA (US)

(72) Inventors: Mitchell SMITH, Redmond, WA (US); David LINVILLE, Redmond, WA (US); Wendy WARNE, Redmond, WA (US); Reza SHARIF, Redmond, WA (US)

(21) Appl. No.: 15/526,720

(22) PCT Filed: Oct. 13, 2015
(86) PCT No.: PCT/US2015/060755

§ 371 Date: May 12, 2017

Related U.S. Application Data
(60) Provisional application No. 62/080,160, filed on Nov. 14, 2014.

(51) Int. Cl. A61B 5/021; A61B 5/08; A61B 5/022

(52) U.S. Cl. CPC A61B 5/02141; A61B 5/022; A61B 5/08

(57) ABSTRACT

Technologies and implementations for a multifunctional healthcare monitoring apparatus are generally disclosed.

### RELATED APPLICATION

This application claims benefit of priority to U.S. Provisional Patent Application Ser. No. 62/080,160 filed on Nov. 14, 2014, titled COMMON MOTOR FOR CAPNOGRAPHY AND NIBP SAMPLING, which is incorporated herein by reference in its entirety.

### INFORMATION

Unless otherwise indicated herein, the approaches described in this section are not prior art to the claims in this application and are not admitted to be prior art by inclusion in this section.

Information about a person's health may be discerned from a wide variety of health related activities of the person. One example of health related activity of the person may include activity related to a person's respiratory system. Another example of health related activity of the person may include activity related to a person's circulatory system. Accordingly, from the activity of the person's respiratory system and/or the activity of the person's circulatory system, information about the person's health may be discerned.

Activity related to a person's respiratory system may provide information of various gases being exchanged by the person. For example, monitoring concentration or partial pressure of gases such as, but not limited to, carbon dioxide (CO₂) in the person's breath may provide a wide variety of information of various gases being exchanged by the person regarding the person's health.

Activity related to a person's circulatory system may include information regarding the person's blood pressure. For example, monitoring blood pressure may also provide a wide variety of information regarding the person's health.

Monitoring of various health related actives may be facilitated by a wide variety of electrical and/or mechanical devices.

### SUMMARY

The present disclosure describes example methods, apparatus, and systems related to a multifunctional healthcare monitoring apparatus. Example apparatus may include motorized driver and a first pump coupled to the motorized driver. The first pump may be configured to produce airflow in a first direction. The example apparatus may also include a second pump coupled to the motorized driver. The second pump may be configured to produce airflow in a second direction, where the airflow in the first direction may be substantially opposite the airflow in the second direction.

The present disclosure describes example methods, where an example method may include a method of operating a multifunctional healthcare monitoring apparatus having a first pump and a second pump. The example method may include engaging a common drive coupling at the second pump. The first pump may be configured to provide substantially continuous pressure. The example method may also include receiving an indication to measure blood pressure and engaging the common drive coupling at the second pump. The second pump may be configured to provide pressure at predetermined intervals to a blood pressure cuff. The example method may include determining if the blood pressure cuff is at or above a person's systolic pressure, and if it is determined that the blood pressure cuff is at or above the person's systolic pressure, disengaging the common drive coupling from the second pump. The example method may include deflating the blood pressure cuff, and determining a systolic pressure and a diastolic pressure based, at least in part, on the deflation of the blood pressure.

The present disclosure describes example machine readable non-transitory medium having stored instructions. The example machine readable non-transitory medium may include instructions that, when executed by one or more processors, operatively enable a pneumatic control module to engage a common drive coupling at a first pump. The first pump may be configured to provide substantially continuous pressure. The example machine readable non-transitory medium may include instructions that, when executed by one or more processors, operatively enable the pneumatic control module to receive an indication to measure blood pressure and to engage the common drive coupling at a second pump. The second pump may be configured to provide pressure at predetermined intervals to a blood pressure cuff. The example machine readable non-transitory medium may include instructions that, when executed by one or more processors, operatively enable the pneumatic control module to determine if the blood pressure cuff is at or above a person's systolic pressure, and if it is determined that the blood pressure cuff is at or above the person's systolic pressure, disengaging the common drive coupling from the second pump. The example machine readable non-transitory medium may include instructions that, when executed by one or more processors, operatively enable the pneumatic control module to deflate the blood pressure cuff, and to determine a systolic pressure and a diastolic pressure based, at least in part, on the deflation of the blood pressure cuff.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIG. 1 illustrates an example multifunctional healthcare monitoring apparatus, in accordance with various embodiments;

FIG. 2 illustrates a block diagram of a blood pressure device in accordance with various embodiments;

FIG. 3 illustrates a block diagram of a gas analysis device in accordance with various embodiments;

FIG. 4 illustrates block diagram of multifunctional healthcare monitoring apparatus in accordance with various embodiments;

FIG. 5 illustrates an operational flow for a multifunctional healthcare monitoring apparatus, arranged in accordance with at least some embodiments described herein;

FIG. 6 illustrates an example computer program product 600, arranged in accordance with at least some embodiments described herein;

FIG. 7 is a block diagram illustrating components of defibrillator device 700, which may be used with various embodiments disclosed herein; and

FIG. 8 is a block diagram illustrating an example computing device 800, such as might be embodied by a person skilled in the art, which is arranged in accordance with at least some embodiments of the present disclosure.

### DETAILED DESCRIPTION

The following description sets forth various examples along with specific details to provide a thorough understanding of claimed subject matter. It will be understood by those skilled in the art, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to methods, apparatus, and systems related to a multifunctional healthcare monitoring apparatus having multiple capabilities.

In healthcare related situations, there may be several indicators of a person's health. For example, one indication of a person's health may include measuring a person's circulatory system such as, but not limited to, a person's blood pressure. Another example may include measuring a person's respiration such as, but not limited to, the concentration of various gases expelled by the person. Another example may include measuring various chemicals a person may expel from the body such as, but not limited to, chemicals during respiration. Accordingly, it may be of no surprise that one of the acronyms a person may be taught when being taught first aid may be “ABC”, which may stand for Airway, Breathing, and Circulation (ABC).

In relation to circulation, a blood pressure measuring and monitoring device (hereon out, a blood pressure device) may facilitate determining perfusion of blood in a person's body (i.e., how well the blood is being delivered to the various parts of the body). In some cases, the blood pressure device may facilitate the diagnosis of potential circulatory issues such as, but not limited to, heart related issues, vital organ related issues, hypertension or hypotension (blood pressure too high or too low), etc. Accordingly, the blood pressure device may facilitate diagnosis of a wide range of health related issues.

In relation to breathing, a breathing measuring and monitoring device may facilitate determining how well the person is being ventilated (i.e., is the person breathing well and exchanging gases appropriately). One example of a breathing measuring and monitoring device may include a capnography device. The capnography device may be capable of monitoring the concentration of CO₂ at the end of each exhaled breath by the person. The concentration of CO₂ at the end of each exhaled breath may be known as end-tidal carbon dioxide (ETCO₂). The capnography device may provide information related to the cardiac output, pulmonary blood flow (i.e., perfusion) as the CO₂ may be transported by the circulatory system to the right side of the heart and then pumped into the lungs by the right ventricle, alveolar ventilation at the lungs, respiratory issues, metabolism, etc. Accordingly, the capnography device may facilitate diagnosis of a wide range of health related issues.

There may be some situations, where both a blood pressure device and a capnography device, among numerous other devices (e.g., pulse oximeter for measuring oxygen saturation or SPO₂), may be employed. One example situation may be in surgery, where the patient may be sedated. In this example, a blood pressure device may monitor and measure the perfusion of blood in the patient, while a capnography device may be used to monitor and measure the ventilation of the patient.

Another example may be in emergency medical services (EMS). An EMS personnel may employ a blood pressure device along with a capnography device. As previously described, the blood pressure device may facilitate measuring and monitoring of circulation of the person, while the capnography device may facilitate measuring and monitoring of ET CO₂. Employing the blood pressure device along with the capnography device may facilitate measuring and monitoring of any necessary cardio pulmonary resuscitation (CPR) activities (e.g., the blood pressure device and/or the capnography device may help determine the effectiveness of the CPR). Additionally, EMS personnel may utilize the capnography device to confirm correct endotracheal tube placement for intubation. Further, the capnography device may facilitate determination of a change in the condition of a person (e.g., on set of shock after injury).

A blood pressure device and a capnography device may both be considered to be pneumatic devices (i.e., air/gas management devices). For the purposes of the present disclosure, air and gas will be referred to generically as “gas”, but it should be appreciated that it is contemplated that the disclosed subject matter includes a wide variety of gases and/or gaseous substances. The blood pressure device may commonly involve management of inflation and deflation of a blood pressure monitor cuff. On the other hand, the capnography device may commonly involve management of a steady flow of exhaled gases for analysis. The blood pressure device may involve a pump, while a capnography device may involve a vacuum (i.e., a pump in reverse) to draw gas. Because of the two differing manners in the way gas may be managed between the two devices, the blood pressure device and the capnography device may commonly be separate devices or managed separately. An integrated device having both blood pressure device capabilities and capnography device capabilities may be beneficial for measuring and monitoring a person's health.

Before moving on to the description of the figures, even though the above may have been mostly described with respect to a blood pressure device and a capnography device, it should be appreciated that it is contemplated within the present disclosure that the claimed subject matter may be applicable to a wide variety of devices, which may or may not utilize pneumatic devices such as, but not limited to, lab-on-chip (LOC) type devices, continuous positive airway pressure (CPAP) type device, and so forth. For example, a micro-electrical-mechanical system device such as, but not limited to, a LOC type device may receive exhaled breath from a person and facilitate analysis of the breath for various diagnostic purposes. Some examples of analysis of the breath for various diagnostic purposes may include a wide variety of analysis such as, but not limited to, chemical analysis, medical diagnostics, pharmaceutics related analysis, immunoassay analysis, nucleic acid based analysis, etc. In the example of a CPAP type device, a CPAP type device may be configured to utilize pneumatic pressures (e.g., positive and/or negative pressures) to provide positive airway pressure to persons requiring such airway pressures such as, but not limited to, persons having breathing problems (e.g., sleep apnea). Accordingly, the claimed subject matter is not limited in scope to the particular implementations described herein.

In a non-limiting example, an integrated apparatus may include a blood pressure device, a capnography device, a CPAP device, and/or a LOC device, or any combination thereof. Additionally, these devices may be integrated as part of emergency equipment such as, but not limited to, a defibrillator device. As will be described in detail, the claimed subject matter may include these configurations and may include much more.

Because the disclosure may encompass a wide variety of healthcare related devices, it is contemplated within the present disclosure that the claimed subject matter is not limited to pneumatic healthcare related devices. For example, any and/or all of the devices previously mentioned may be included as a component of an electrical healthcare device such as, but not limited to, a defibrillator type device. For example, a defibrillator type device may include an external defibrillator type device. The external defibrillator device may include a defibrillator device intended to treat a limited number of people such as, but not limited to, a single person. Single person type external defibrillators may include relatively small (i.e., portable) external defibrillator devices. An example of a single person type external defibrillator may be an automated external defibrillator (AED) type device. AED type devices may be found in various private and/or public places such as, but not limited to, offices, train stations, airports, stadiums, hospitals, homes, vehicles, vessels, planes, trains, automobile, etc. AED type devices may be commonly for use by a layperson and/or a person with basic life support training.

Another example type of external defibrillator device may include wearable defibrillator devices, which may be worn outside the body. Wearable defibrillator devices may continuously monitor a person's heart with electrodes capable of sensing to detect VF or other heart arrhythmia. Wearable defibrillator devices may provide an intermediate care option for a person having a high risk of a coronary heart event and/or a person who may not be a candidate for an Implantable Cardioverter Defibrillator (ICD). Accordingly, the claimed subject matter is not limited in scope to any particular implementation described herein.

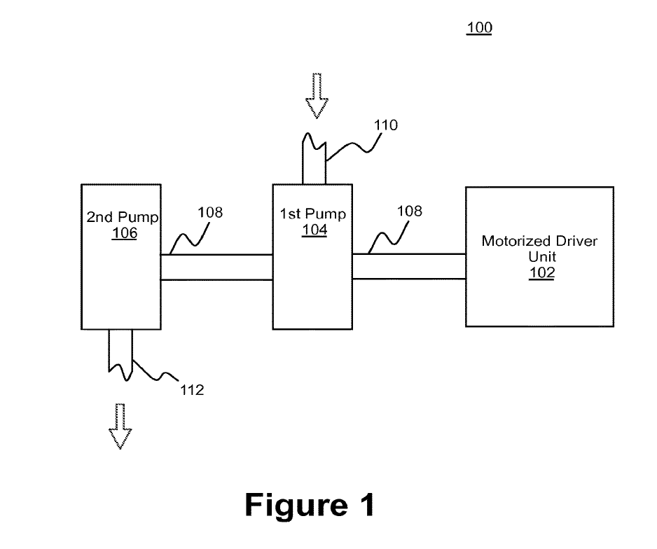

FIG. 1 illustrates an example multifunctional healthcare monitoring apparatus, in accordance with various embodiments. Shown in FIG. 1, the multifunctional healthcare monitoring apparatus 100 may comprise a motorized driver unit 102, a first pump 104 coupled to the motorized driver 102, and a second pump 106 coupled to the motorized driver 102. As shown, the second pump 106 may be coupled to the motorized driver 102 via the first pump 104. Accordingly, the first pump 104 and the second pump 106 may share a common drive coupling 108. In FIG. 1, the motorized driver unit 102 may provide drive power to the first pump 104 and to the second pump 106 via the common drive coupling. As shown, the first pump 104 and the second pump 106 being coupled to the motorized driver unit 102 via the common drive coupling 108 may facilitate multifunctional healthcare monitoring of a person.

In one example, the first pump 104 may be driven by the motorized driver unit 102 to produce a negative pressure (i.e., vacuum) to facilitate drawing of gas from a person into an inlet 110 (e.g., the first pump 104 may have functionality as a capnography type device). Accordingly, the first pump 104 may facilitate measuring and monitoring the breathing of the person as previously described.

In another example, the second pump 106 may be driven by the motorized driver unit 102 to produce a positive pressure to facilitate pumping of gas out of an outlet 112 (e.g., the second pump 106 may have functionality as a blood pressure device). Accordingly, the second pump 106 may facilitate determining perfusion of blood in the person's body (i.e., how well the blood is being delivered to the various parts of the body) as previously described.

In FIG. 1, the motorized driver unit 102 may be configured to provide power to the first pump 104 and the second pump 106 via the common drive coupling 108. However, as will be described in further detail, the motorized driver unit 102 and the common drive coupling 108 may be configured to provide power to one of the first pump 104 or the second pump 106 and/or power to both the first pump 104 and the second pump 106. For example, the first pump 104 may be configured to be utilized as a capnography device, and accordingly, the first pump 104 may operate to substantially continuously draw gas at a predetermined rate such as, but not limited to, approximately, 150 mL/minute. As the first pump 104 may operate substantially continuously, the second pump 106 may be configured to operate a blood pressure device, and accordingly, the second pump 106 may operate to inflate a blood pressure cuff at predetermined intervals (e.g., the second pump unit 106 may operate to inflate the blood pressure cuff to a predetermined pressure, and when the predetermined pressure is reached, the second pump 106 may stop).

The second pump 106 may operate at predetermined intervals for monitoring and measuring. For example, the intervals may range from every 30 seconds (e.g., when a person may be critically ill or unstable), every 5 minutes to 10 minutes (e.g., when a person is under sedation and/or undergoing some medical procedure), and every 1 hour to 4 hours (e.g., when a person is undergoing normal observation with low urgency).

In one example, in order to facilitate the second pump 106 to operate at predetermined intervals, the common drive coupling 108 may be configured to engage and disengage the second pump 106. For example, the common drive coupling 108 may be configured to engage and disengage a pump mechanism such as, but not limited to, an impeller, at the second pump 106. The first pump unit 104 may continue to operate via the common drive coupling, while the second pump unit 106 operates at predetermined intervals.

In one example, the multifunctional healthcare monitoring apparatus 100 may comprise a direct current (DC) micro motor coupled to the motorized driver unit 102. The micro motor may comprise a DC micro motor having a precious metal commutator.

In one example, the common drive coupling 108 of the multifunctional healthcare monitoring apparatus 100 may comprise a motorized drive shaft coupling the motorized driver unit 102 to the first pump 104 and the second pump 106. In another example, the common drive coupling 108 of the multifunctional healthcare monitoring apparatus 100 may comprise a reciprocating drive shaft. In another example, the second pump 106 may be configured to facilitate non-invasive blood pressure (NIBP) monitoring functionality. In yet another example, the first pump 104 may comprise a pump configured to facilitate chemical analysis utilizing micro-electro-mechanical systems (MEMS) devices. An example of an apparatus configured to facilitate chemical analysis utilizing micro-electro-mechanical systems (MEMS) devices may be an apparatus comprising a lab-on-chip (LOC) device.

In another example, the multifunctional healthcare monitoring apparatus 100 may comprise a first pump 104 configured to facilitate continuous positive airway pressure (CPAP) functionality.

In one example, the first pump 104 and/or the second pump 106 may include a wide variety of pumping mechanisms such as, but not limited to, a piston type mechanism, an impeller type mechanism, a fan type mechanism, and so forth, and any combination thereof. In another example, the first pump 104 and/or the second pump 106 may be of a wide variety of pumps such as, but not limited to, a positive displacement type pump, a momentum transfer type pump, or an entrapment type pump.

As described, the multifunctional healthcare monitoring apparatus 100 may include a wide variety of healthcare monitoring apparatus including a wide variety of pneumatic type devices and/or a wide variety of mass transport type device, and accordingly, the claimed subject matter is not limited in these respects. Additionally, multifunctional healthcare monitoring apparatus 100 may be included in a wide range of form factors such as, but not limited to, miniature devices. Accordingly, any of the components described herein may include miniature components and/or micro components. For example, any or all of components (e.g., the motorized drive unit 102, the common drive coupling 108, the first pump 104, and the second pump 106) may be implemented utilizing micro drive technology.

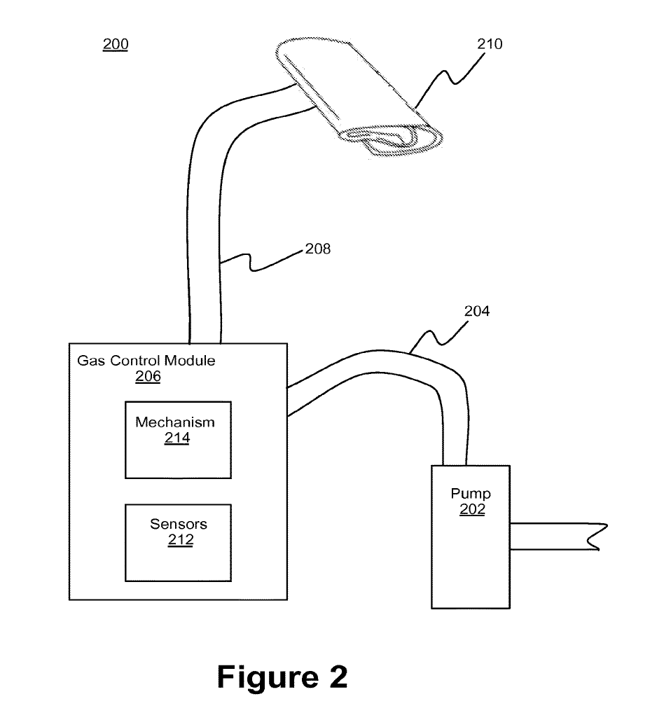

FIG. 2 illustrates a block diagram of a blood pressure device in accordance with various embodiments. In FIG. 2, a blood pressure device 200 may include a pump 202, a first gas coupling 204, a gas control module 206, a second gas coupling 208, and a blood pressure cuff 210. The pump 202 may be similar to the second pump 106 as described with respect to FIG. 1. Accordingly, the pump 202 may be driven by the common drive coupling 108.

As shown in FIG. 2, the first gas coupling may facilitate passage of gas between the pump 202 and the gas control module 206, and the first gas coupling may facilitate passage of gas between the gas control module 206 and the blood pressure cuff 210. In one example, the blood pressure device 200 may be configured to be a non-invasive blood pressure (NIBP) measuring and monitoring type device. Accordingly, the blood pressure device 200 may include sensors 212 and/or mechanisms 214 to facilitate oscillometric functionality for the blood pressure device 200. For example, the blood pressure device 200 may be configured to determine an amplitude of a person's pulse as the blood pressure cuff 210 is deflated from above a systolic pressure, determine a sudden increase in the amplitude, determine a diastolic pressure based, at least in part, on a transition of the amplitude from a maximum. Additionally, the mechanisms 214 of the gas control module 206 may include various mechanisms such as, but not limited to, various gas channels, various gas bleed mechanisms, various gas valves, and so forth to facilitate NIBP measuring and monitoring. Additionally, the blood pressure device 200 may include sensors 212 and/or mechanisms 214 to facilitate auscultatoric functionality for the blood pressure device 200. For example, the mechanisms 214 of the blood pressure device 200 may include audio sensors (not shown) to help facilitate detection of flow of blood, which may be referred to as Korotkoff sounds. The Korotkoff sounds may help facilitate determination of systolic blood pressure and diastolic arterial pressure.

As previously described, the blood pressure device 200 may operate at predetermined intervals (e.g., various periods, cycles, etc.). Accordingly, the pump 202 may be configured to engage and disengage from the common drive coupling 108 at the predetermined intervals.

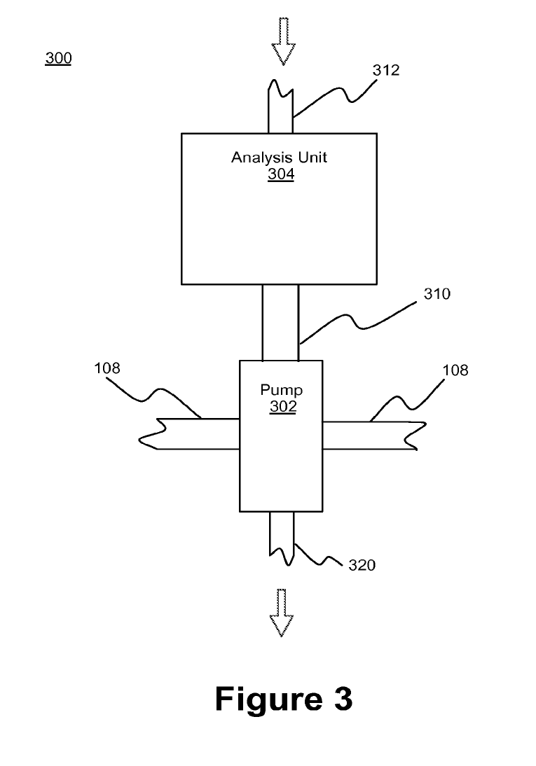

FIG. 3 illustrates a block diagram of a gas analysis device in accordance with various embodiments. In FIG. 3, a gas analysis device (here on out, “analyzer”) 300 may include a pump 302. The pump 302 may be similar to the first pump 104 as described with respect to FIG. 1. Additionally, the analyzer 300 may include an analysis unit 304. The analysis unit 304 may be coupled to the pump 302 via a gas coupling 310. Additionally, the analysis unit 304 may have a gas inlet 312 to facilitate drawing of gas into the analysis unit 304. As shown, the common drive coupling 108 as described with respect to FIG. 1 may drive the pump 302.

In one non-limiting example, the analyzer 300 may be configured to be a capnography type device. For the capnography type device example, the pump 302 may be a pump configured to develop a negative pressure (i.e., a vacuum). Accordingly, the pump 302 may be configured to draw gas into the analysis unit 304 from the gas inlet 312. The gas may include exhaled gas from a person, where the exhaled gas may be received at the analysis unit 304 via the intake 312.

Continuing with the example of the capnography type device, the analysis unit 304 may receive the exhaled gas from the person and be configured to determine a concentration of CO₂ gas in the person's exhaled breath. The concentration may be determined by utilization of infrared analysis. In order to facilitate the infrared analysis, the pump 302 may be configured to draw gas at a rate conducive to such analysis such as, but not limited to, approximately 50 mL/minute to 150 mL/minute with the rate being able to be adjusted based upon the person's need (e.g., neonatal and pediatric).

In FIG. 3, the analyzer 300 may be configured to facilitate a capnography type device. However, the analyzer 300 may be configured to be a wide variety of pneumatic type devices as contemplated within the scope of the claimed subject matter. In one example, the analyzer 300 may be configured to be a LOC type device. The pump 302 may be configured to draw gas from a wide variety of sources such as, but not limited to, exhaled gas from a person, the atmosphere, enclosed space, open space, a container, etc. The drawn gas may be received at the analysis unit 304, where the analysis unit 304 may be configured to analyze the received gas for a wide variety of chemical analysis such as, but not limited to, medical diagnostics, pharmaceutics related analysis, immunoassay analysis, nucleic acid based analysis as previously described.

In another example, the analyzer 300 may be configured to be a CPAP type device. The pump 302 may be configured to draw in gas from the atmosphere and provide the positive airflow to a person via an outlet 310. Continuing with the example of the CPAP type device, it may be appreciated that the pump 302 may be configured to provide gas at pressures above atmospheric pressures such as, but not limited to, approximately 3 mmHg to 15 mmHg having gas flow rates such as, but not limited to approximately 20 L/minute to 60 L/minute.

Briefly turning back to FIG. 1, since the multifunctional healthcare monitoring apparatus 100 may include two pumps, the first pump 104 and the second pump 106, in FIG. 3., the pump 302 may be configured to draw gas and provide the drawn gas after analysis of some kind to the pump 206 shown in FIG. 2. Accordingly, in one example, the multifunctional healthcare monitoring apparatus 100 may approximately be a closed pneumatic circuit. The multifunctional healthcare monitoring apparatus 100 may include the blood pressure device 200 and the analyzer 300, both configured to be driven by the motorized drive unit 102.

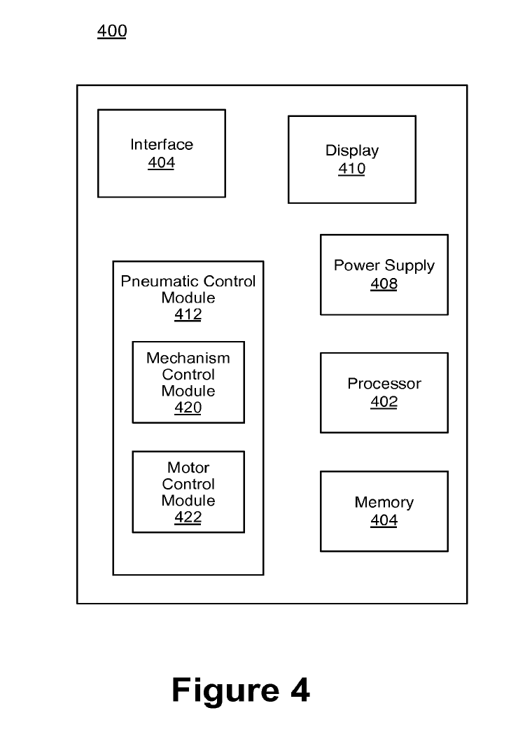

FIG. 4 illustrates block diagram of multifunctional healthcare monitoring apparatus in accordance with various embodiments. In FIG. 4, a multifunctional healthcare monitoring apparatus 400 may include a processor 402, an interface module 404, a memory 406, a power supply 408, a display 410, and a pneumatic control module (hereon out, PC module) 412. The various components, the processor 402, the interface module 404, the memory 406, the display 410, and the PC module 412 may be communicatively coupled with one another. For example, the PC module 412 may be communicatively coupled to the processor 402 and/or the memory 406. In order to operate, the multifunctional healthcare monitoring apparatus 400 may receive power from the power supply 408. Additionally shown, the PC module 412 may include a mechanism control module (hereon out, MC module) 420 and/or a motor control module (hereon out, MOC module) 422. Accordingly, the PC module 412 may be configured to facilitate control of the various mechanisms, as previously described with respect to FIG. 2, associated with the multifunctional healthcare monitoring apparatus 100, and/or configured to facilitate control of the motorized drive unit 102, which in turn, may control the common drive coupling 108 (shown in FIG. 1).

The memory 406 may have stored various information such as, but not limited to, pressure operating ranges, oscillometric information, auscultatoric information, various information received from sensors, which may be included in the multifunctional healthcare monitoring apparatus 100, etc.

The interface module 404 may facilitate interface functionalities for the multifunctional healthcare monitoring apparatus 100. For example, the interface module 404 may facilitate input of various control prescriptions such as, but not limited to, pressure operating ranges by healthcare personnel. The interface module 404 may be touch screen and/or physical keys (e.g., integrated with the display 410).

The multifunctional healthcare monitoring apparatus 400 may facilitate control and functionalities for multifunctional healthcare monitoring as described with respect to FIGS. 1-3.

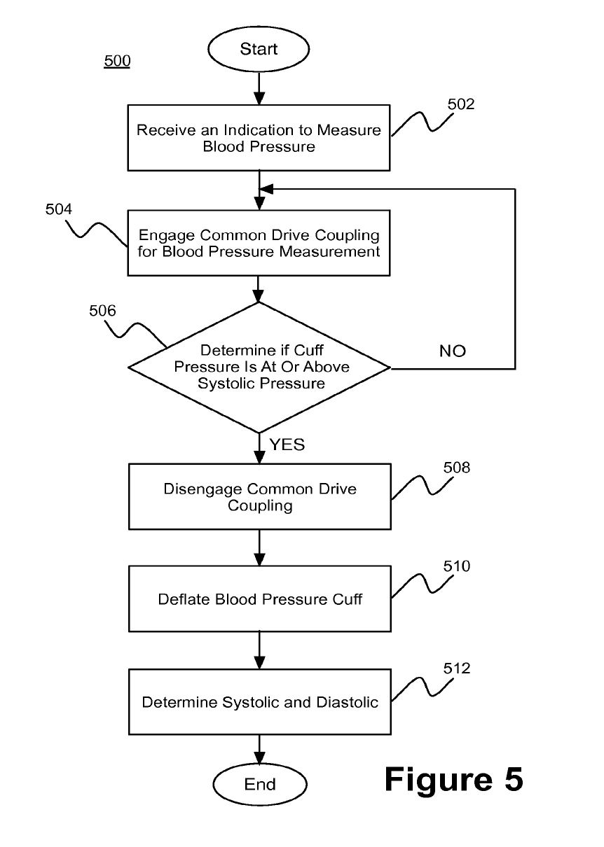

FIG. 5 illustrates an operational flow for a multifunctional healthcare monitoring apparatus, arranged in accordance with at least some embodiments described herein. In some portions of the description, illustrative implementations of the method are described with reference to the elements of the components described with respect to FIGS. 1-4. However, the described embodiments are not limited to these depictions. More specifically, some elements depicted in FIGS. 1-4 may be omitted from some implementations of the methods details herein. Furthermore, other elements not depicted in FIGS. 1-4 may be used to implement example methods detailed herein.

Additionally, FIG. 5 employs block diagrams to illustrate the example methods detailed therein. These block diagrams may set out various functional block or actions that may be described as processing steps, functional operations, events and/or acts, etc., and may be performed by hardware, software, and/or firmware. Numerous alternatives to the functional blocks detailed may be practiced in various implementations. For example, intervening actions not shown in the figures and/or additional actions not shown in the figures may be employed and/or some of the actions shown in one figure may be operated using techniques discussed with respect to another figure. Additionally, in some examples, the actions shown in these figures may be operated using parallel processing techniques. The above described, and other not described, rearrangements, substitutions, changes, modifications, etc., may be made without departing from the scope of the claimed subject matter.

In some examples, operational flow 500 may be employed as part of a multifunctional healthcare monitoring apparatus. As previously described, the multifunctional healthcare monitoring apparatus may include a first pump and a second pump. The first pump and the second pump may share a common drive coupling. The common drive coupling may be engaged at the first pump to provide substantially continuous pressure.

Beginning at block 502 (“Receive an Indication to Measure Blood Pressure”), the PC module 412 (shown in FIG. 4) may receive an indication to initiate a blood pressure measurement (e.g., NIBP). As described, the PC module 412 may include the MC module 420 and/or the MOC module 422. The MC module 420 and/or the MOC module 422 may be utilized to control at least some of the functionality as described with respect to FIGS. 1-4 (e.g., blood pressure device and/or capnography device).

Continuing from block 502 to 504 (“Engage Common Drive Coupling for Blood Pressure Measurement”), under the control of the PC module 412, the motorized driver unit 102 may provide power to the second pump 106 via the common drive coupling 108. The second pump 106 may be engaged as a blood pressure device as described with respect to the FIG. 2. and may provide pressure at predetermined intervals to the blood pressure cuff 210.

Continuing from block 504 to decision diamond 506 (“Determine if Cuff Pressure Is At Or Above Systolic Pressure”), various sensors may facilitate oscillometric functionality and/or auscultatoric functionality. Accordingly, referring to FIG. 2, the pump 202 may pump gas into the blood pressure cuff 210 until the systolic pressure is reached.

In one example, if it is determined that the systolic pressure is reached, the gas being pumped into the blood pressure cuff 210 would be stopped. The gas control module 206 may achieve the stopping of the gas into the blood pressure cuff 210 by disengaging the common drive coupling 108 from the second pump 106 at block 508 (“Disengage Common Drive Coupling”). However, if it is determined that the systolic pressure is not yet reached, the gas control module 206 may continue to engage the common drive coupling to provide power to the second pump 106.

Once the second pump 106 is disengaged from the common drive coupling 108, the gas control module 206 may facilitate a carefully controlled deflation of the blood pressure cuff 210 at block 510 (“Deflate Blood Pressure Cuff”). Continuing from block 510 to 512 (“Determine Systolic and Diastolic”), various sensors may facilitate oscillometric functionality and/or auscultatoric functionality to determine the person's systolic and diastolic measurements.

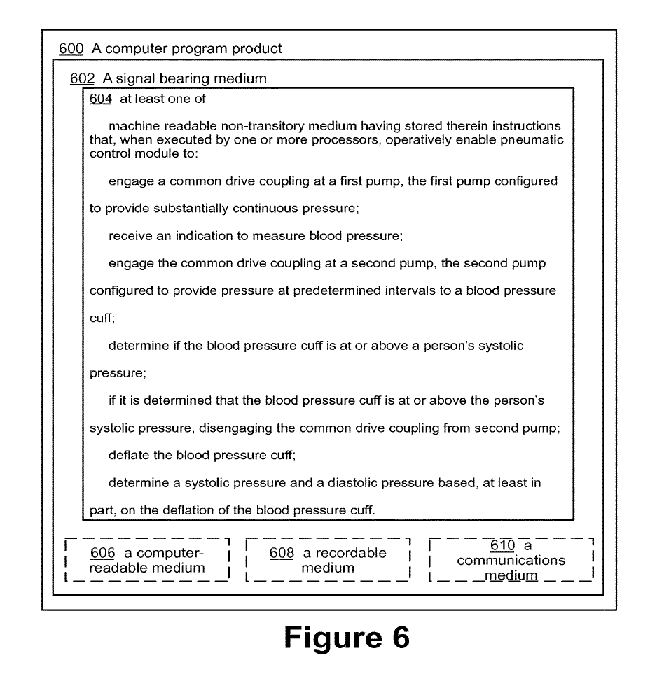

In general, the operational flow described with respect to FIG. 5 and elsewhere herein may be implemented as a computer program product, executable on any suitable computing system, or the like. For example, a computer program product for facilitating utilization of a multifunctional healthcare monitoring apparatus. Example computer program products may be described with respect to FIG. 6 and elsewhere herein.

FIG. 6 illustrates an example computer program product 600, arranged in accordance with at least some embodiments described herein. Computer program product 600 may include machine readable non-transitory medium having stored therein instructions that, when executed, cause the machine to utilize multifunctional healthcare monitoring apparatus, according to the processes and methods discussed herein. Computer program product 600 may include a signal bearing medium 602. Signal bearing medium 602 may include one or more machine-readable instructions 604, which, when executed by one or more processors, may operatively enable a computing device to provide the functionality described herein. In various examples, the devices discussed herein may use some or all of the machine-readable instructions.

In some examples, the machine readable instructions 604 may include instructions that, when executed, cause the machine to engage a common drive coupling at a first pump, the first pump configured to provide substantially continuous pressure. In some examples, the machine readable instructions 604 may include instructions that, when executed, cause the machine to receive an indication to measure blood pressure. In some examples, the machine readable instructions 604 may include instructions that, when executed, cause the machine to engage the common drive coupling at a second pump, the second pump configured to provide pressure at predetermined intervals to a blood pressure cuff. In some examples, the machine readable instructions 604 may include instructions that, when executed, cause the machine to determine if the blood pressure cuff is above a person's systolic pressure. In some examples, the machine readable instructions 604 may include instructions that, when executed, cause the machine to disengage the common drive coupling from the second pump if it is determined that the blood pressure cuff is at or above the person's systolic pressure. In some examples, the machine readable instructions 604 may include instructions, that when executed, may operate to deflate the blood pressure cuff. In some examples, the machine readable instructions 604 may include instructions that, when executed, cause the machine to determine a systolic pressure and a diastolic pressure based at least in part, on the deflation of the blood pressure cuff.

In some implementations, signal bearing medium 602 may encompass a computer-readable medium 606, such as, but not limited to, a hard disk drive, a Compact Disc (CD), a Digital Versatile Disk (DVD), a digital tape, memory, etc. In some implementations, the signal bearing medium 602 may encompass a recordable medium such as, but not limited to, memory, read/write (R/W) CDs, R/W DVDs, etc. In some implementations, the signal bearing medium 602 may encompass a communications medium such as, but not limited to, a digital and/or an analog communication medium (e.g., a fiber optic cable, a waveguide, a wired communication link, a wireless communication link, etc.). In some examples, the signal bearing medium 602 may encompass a machine readable non-transitory medium.

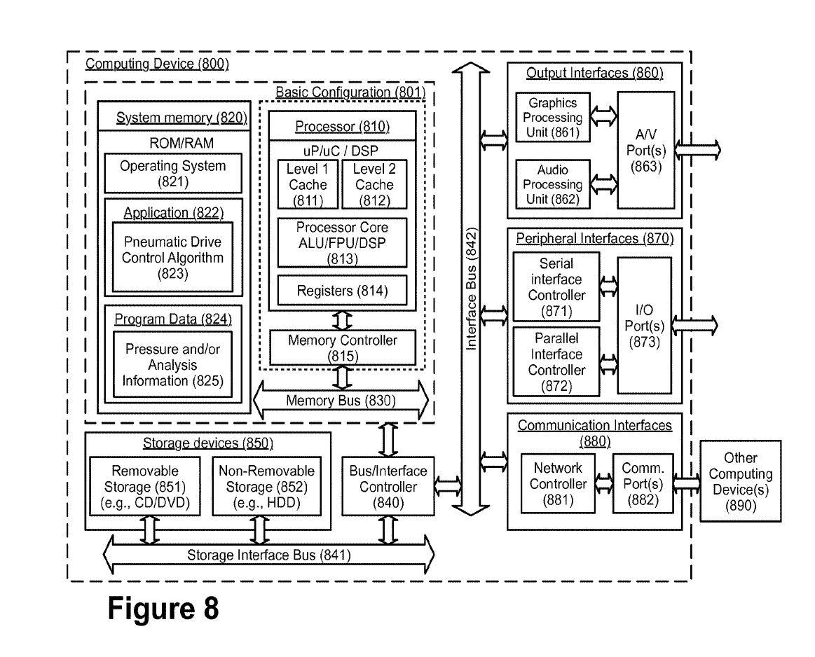

In general, the methods described with respect to FIG. 6 and elsewhere herein may be implemented in any suitable computing system. Example systems may be described with respect to FIG. 8 and elsewhere herein. In general, the system may be configured to facilitate utilization of a multifunctional healthcare monitoring apparatus.

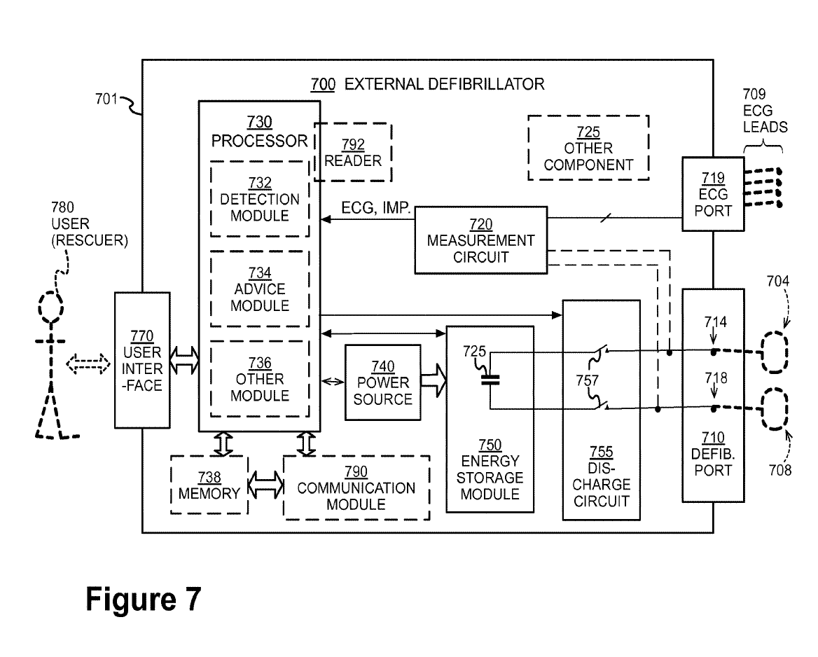

FIG. 7 is a block diagram illustrating components of defibrillator device 700, which may be used with various embodiments disclosed herein. These components may be, for example, integrated with the various components and embodiments described with respect to FIGS. 1-4. The components of FIG. 7. may be provided in a housing 701, which may be known as casing 701.

The defibrillator device 700 may be intended for use by a user 780 (e.g., a rescuer). The defibrillator device 700 may typically include a defibrillation port 710, such as a socket in housing 701. The defibrillation port 710 may include nodes 714 and 718. One or more electrodes 704 and 708 may be plugged in to the defibrillation port 710, so as to make electrical contact with nodes 714 and 718, respectively. It may also be possible that the electrodes 704 and 708 may be connected continuously to the defibrillation port 710, etc. Either way, the defibrillation port 710 may be used for guiding via the electrodes 704 and 708 to a person an electrical charge that may have been stored in the defibrillator device 700, as described herein.

If the defibrillator device 700 comprise of a defibrillator-monitor and the defibrillator device 700 may also have an ECG port 719 in the housing 701, for receiving ECG leads 709. The ECG leads 709 may facilitate sensing of an ECG signal (e.g., a 12-lead signal or from a different number of lead signals). Moreover, a defibrillator-monitor could have additional ports (not shown), and the other component 725 may be configured to filter the ECG signal (e.g., application of at least one filter to the signal to help facilitate removal of artifacts such as, but not limited to, chest compression due to chest compressions being delivered to the person).

The defibrillator 700 also may include a measurement circuit 720. The measurement circuit 720 may receive physiological signals from the ECG port 719, and also from other ports, if provided. The circuit 720 may render detected physiological signals and their corresponding information. The information may be in the form of data, or other signals, etc.

If the defibrillator 700 is configures as an AED type device, ECG port 719 may not be present. The measurement circuit 720 may obtain physiological signals through the nodes 714 and 718 instead, when the electrodes 704 and 708 are attached to the person, as previously described. In these cases, a person's ECG signal may be detected as a voltage difference between the electrodes 704 and 708. Additionally, the impedance between the electrodes 704 and 708 may be detected, among other things, whether the electrodes 704 and 708 have been inadvertently disconnected from the person.

The defibrillator 700 may also include a processor 730. The processor 730 may be implemented in a wide variety of manners for causing actions and operations to be performed. Some examples may include digital and/or analog processors such as microprocessors and digital-signal processors (DSPs), controllers such as microcontrollers, software running in a machine environment, programmable circuits such as Field Programmable Gate Arrays (FPGAs), Field-Programmable Analog Arrays (FPAAs), Programmable Logic Devices (PLDs), Application Specific Integrated Circuits (ASICs), and so on or any combination thereof.

The processor 730 may include a number of modules. One example module may be a detection module 732, which may detect outputs from the measurement circuit 720. The detection module 732 may include a VF detector. Accordingly, the person's detected ECG may be utilized to help determine whether the person is experiencing VF.

In another example module may be an advice module 734, which may provide advice based, at least in part, on outputs of detection module 732. The advice module 734 may include an algorithm such as, but not limited to, Shock Advisory Algorithm, implement decision rules, and so on. For example, the advice may be to shock, to not shock, to administer other forms of therapy, and so on. If the advice is to shock, some defibrillator examples may report the advice to the user, and prompt them to do it. In other examples, the defibrillator device may execute the advice by administering the shock. If the advice is to administer CPR, the defibrillator 700 may further issue prompts for administrating CPR, and so forth.

The processor 730 may include additional modules, such as module 736 for various other functions. Additionally, if other component 725 is provided, it may be operated in part by processor 730, etc.

In an example, the defibrillator device 700 may include a memory 738, which may work together with the processor 730. The memory 738 may be implemented in a wide variety of manners. For example, the memory 738 may be implemented such as, but not limited to, nonvolatile memories (NVM), read-only memories (ROM), random access memories (RAM), and so forth or any combination thereof. The memory 738 may can include programs for the processor 730, and so on. The programs may include operational programs execution by the processor 730 and may also include protocols and methodologies that decisions may be made by advice module 734. Additionally, the memory 738 may store various prompts for the user 780, etc. Moreover, the memory 738 may store a wide variety of information (i.e., data) such as, but not limited to information regarding the person.

The defibrillator 700 may also include a power source 740. In order to facilitate portability of defibrillator device 700, the power source 740 may include a battery type device. A battery type device may be implemented as a battery pack, which may be rechargeable or not be rechargeable. At times, a combination of rechargeable and non-rechargeable battery packs may be utilized. Examples of power source 740 may include AC power override, where AC power may be available, and so on. In some examples, the processor 730 may control the power source 740.

Additionally, the defibrillator device 700 may include an energy storage module 750. The energy storage module 750 may be configured to store some electrical energy (e.g., when preparing for sudden discharge to administer a shock). The energy storage module 750 may be charged from the power source 740 to an appropriate level of energy, as may be controlled by the processor 730. In some implementations, the energy storage module 750 may include one or more capacitors 752, and the like.

The defibrillator 700 may include a discharge circuit 755. The discharge circuit 755 may be controlled to facilitate discharging of the energy stored in energy storage module 750 to the nodes 714 and 718, and also to electrodes 704 and 708. The discharge circuit 755 may include one or more switches 757. The one or more switches 757 may be configured in a number of manners such as, but not limited to, an H-bridge, and so forth.

The defibrillator device 700 may further include a user interface 770 for the user 780. The user interface 770 may be implemented in a variety of manners. For example, the user interface 770 may include a display screen capable of displaying what is detected and measured, provide visual feedback to the user 780 for their resuscitation attempts, and so forth. The user interface 770 may also include an audio output such as, but not limited to, a speaker to issue audio prompts, etc. The user interface 770 may additionally include various control devices such as, but not limited to, pushbuttons, keyboards, switches, track pads, and so forth. Additionally, the discharge circuit 755 may be controlled by the processor 730 or directly by the user 780 via the user interface 770, and so forth.

Additionally, the defibrillator device 700 may include other components. For example, a communication module 790 may be provided for communicating with other machines 100, 200, and 300, as previously described. Such communication may be performed wirelessly, or via wire, or by infrared communication, and so forth. Accordingly, information may be communicated, such as person data, incident information, therapy attempted, CPR performance, ECG information, and so forth.

A feature of a defibrillator device may be CPR related prompting. CPR prompts may be issued to the user 780 visually or by audio facilitating assistance in the administration of CPR by the user 780. Examples may be found in U.S. Pat. No. 6,334,070 and U.S. Pat. No. 6,356,785.

FIG. 8 is a block diagram illustrating an example computing device 800, such as might be embodied by a person skilled in the art, which is arranged in accordance with at least some embodiments of the present disclosure. In one example configuration 801, computing device 800 may include one or more processors 810 and system memory 820. A memory bus 830 may be used for communicating between the processor 810 and the system memory 820.

Depending on the desired configuration, processor 810 may be of any type including but not limited to a microprocessor (μP), a microcontroller (μC), a digital signal processor (DSP), or any combination thereof. Processor 810 may include one or more levels of caching, such as a level one cache 811 and a level two cache 812, a processor core 813, and registers 814. The processor core 813 may include an arithmetic logic unit (ALU), a floating point unit (FPU), a digital signal processing core (DSP Core), or any combination thereof. A memory controller 815 may also be used with the processor 810, or in some implementations the memory controller 815 may be an internal part of the processor 810.

Depending on the desired configuration, the system memory 820 may be of any type including but not limited to volatile memory (such as RAM), non-volatile memory (such as ROM, flash memory, etc.) or any combination thereof. System memory 820 may include an operating system 821, one or more applications 822, and program data 824. Application 822 may include pneumatic drive control algorithm 823 that is arranged to perform the functions as described herein including the functional blocks and/or actions described. Program Data 824 may include, among many information described, pressure and/or analysis related information 825 for use with pneumatic drive control algorithm 823. In some example embodiments, application 822 may be arranged to operate with program data 824 on an operating system 821 such that implementations of a multifunctional healthcare monitoring apparatus may be provided as described herein. For example, apparatus described in the present disclosure may comprise all or a portion of computing device 800 and be capable of performing all or a portion of application 822 such that implementations of multifunctional healthcare monitoring apparatus may be provided as described herein. This described basic configuration is illustrated in FIG. 8 by those components within dashed line 801.

Computing device 800 may have additional features or functionality, and additional interfaces to facilitate communications between the basic configuration 801 and any required devices and interfaces. For example, a bus/interface controller 840 may be used to facilitate communications between the basic configuration 801 and one or more data storage devices 850 via a storage interface bus 841. The data storage devices 850 may be removable storage devices 851, non-removable storage devices 852, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 820, removable storage 851 and non-removable storage 852 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 800. Any such computer storage media may be part of device 800.

Computing device 800 may also include an interface bus 842 for facilitating communication from various interface devices (e.g., output interfaces, peripheral interfaces, and communication interfaces) to the basic configuration 801 via the bus/interface controller 840. Example output interfaces 860 may include a graphics processing unit 861 and an audio processing unit 862, which may be configured to communicate to various external devices such as a display or speakers via one or more A/V ports 863. Example peripheral interfaces 860 may include a serial interface controller 871 or a parallel interface controller 872, which may be configured to communicate with external devices such as input devices (e.g., keyboard, mouse, pen, voice input device, touch input device, etc.) or other peripheral devices (e.g., printer, scanner, etc.) via one or more I/O ports 873. An example communication interface 880 includes a network controller 881, which may be arranged to facilitate communications with one or more other computing devices 890 over a network communication via one or more communication ports 882. A communication connection is one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), infrared (IR) and other wireless media. The term computer readable media as used herein may include both storage media and communication media.

Computing device 800 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 800 may also be implemented as a personal computer including both laptop computer and non-laptop computer configurations. In addition, computing device 800 may be implemented as part of a wireless base station or other wireless system or device.

Some portions of the foregoing detailed description are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is here, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing device, that manipulates or transforms data represented as physical electronic or magnetic quantities within memories, registers, or other information storage devices, transmission devices, or display devices of the computing device.

Claimed subject matter is not limited in scope to the particular implementations described herein. For example, some implementations may be in hardware, such as employed to operate on a device or combination of devices, for example, whereas other implementations may be in software and/or firmware. Likewise, although claimed subject matter is not limited in scope in this respect, some implementations may include one or more articles, such as a signal bearing medium, a storage medium and/or storage media. This storage media, such as CD-ROMs, computer disks, flash memory, or the like, for example, may have instructions stored thereon, that, when executed by a computing device, such as a computing system, computing platform, or other system, for example, may result in execution of a processor in accordance with claimed subject matter, such as one of the implementations previously described, for example. As one possibility, a computing device may include one or more processing units or processors, one or more input/output devices, such as a display, a keyboard and/or a mouse, and one or more memories, such as static random access memory, dynamic random access memory, flash memory, and/or a hard drive.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a flexible disk, a hard disk drive (HDD), a Compact Disc (CD), a Digital Versatile Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter also may include all implementations falling within the scope of the appended claims, and equivalents thereof.

What is claimed:

1. A multifunctional healthcare monitoring apparatus comprising:
a motorized driver;
a first pump coupled to the motorized driver, the first pump configured to produce airflow in a first direction; and
a second pump coupled to the motorized driver, the second pump configured to produce airflow in a second direction, the airflow in the first direction being substantially opposite the airflow in the second direction.

2. The multifunctional healthcare monitoring apparatus of claim 1 further comprising a direct current (DC) micro motor coupled to the motorized driver.

3. The multifunctional healthcare monitoring apparatus of claim 2, wherein the DC micro motor comprises a DC micro motor having a precious metal commutator.

4. The multifunctional healthcare monitoring apparatus of claim 1, wherein the motorized driver comprises a motorized drive shaft.

5. The multifunctional healthcare monitoring apparatus of claim 1, wherein the motorized driver comprises a reciprocating drive shaft.

6. The multifunctional healthcare monitoring apparatus of claim 1, wherein the first pump comprises a pump configured to facilitate capnography functionality.

7. The multifunctional healthcare monitoring apparatus of claim 1, wherein the second pump comprises a pump configured to facilitate non-invasive blood pressure (NIBP) monitoring functionality.

8. The multifunctional healthcare monitoring apparatus of claim 1, wherein the first pump comprises a pump configured to facilitate chemical analysis utilizing micro-electro-mechanical systems (MEMS) devices.

9. The multifunctional healthcare monitoring apparatus of claim 8 further comprising a lab-on-chip (LOC) device.

10. The multifunctional healthcare monitoring apparatus of claim 1, wherein the second pump comprises a pump configured to facilitate continuous positive airway pressure (CPAP) functionality.

11. The multifunctional healthcare monitoring apparatus of claim 1, wherein the first pump comprises at least one of a pump having a piston or a pump having an impeller.

12. The multifunctional healthcare monitoring apparatus of claim 1, wherein the second pump comprises at least one of a pump having a piston or a pump having an impeller.

13. The multifunctional healthcare monitoring apparatus of claim 1, wherein the first pump and/or the second pump comprises at least one of a positive displacement type vacuum pump, a momentum transfer type vacuum pump, or an entrapment type vacuum pump.

14. A method of operating a multifunctional healthcare monitoring apparatus having a first pump and a second pump, the method comprising:
engaging a common drive coupling at the first pump, the first pump configured to provide substantially continuous pressure;
receiving an indication to measure blood pressure;
engaging the common drive coupling at the second pump, the second pump configured to provide pressure at predetermined intervals to a blood pressure cuff;
determining if the blood pressure cuff is at or above a person's systolic pressure;
if it is determined that the blood pressure cuff is at or above the person's systolic pressure, disengaging the common drive coupling from the second pump;
deflating the blood pressure cuff; and
determining a systolic pressure and a diastolic pressure based, at least in part, on the deflation of the blood pressure cuff.

15. The method of claim 14, wherein engaging the common drive coupling at the first pump comprises engaging the common drive coupling to facilitate capnography functionality.

16. The method of claim 14, wherein receiving the indication to measure blood pressure comprises receiving the indication from a pneumatic control module.

17. A machine readable non-transitory medium having stored therein instructions that, when executed by one or more processors, operatively enable a pneumatic control module to:
engage a common drive coupling at a first pump, the first pump configured to provide substantially continuous pressure;
receive an indication to measure blood pressure;
engage the common drive coupling at a second pump, the second pump configured to provide pressure at predetermined intervals to a blood pressure cuff;
determine if the blood pressure cuff is at or above a person's systolic pressure;
if it is determined that the blood pressure cuff is at or above the person's systolic pressure, disengaging the common drive coupling from second pump;
deflate the blood pressure cuff; and
determine a systolic pressure and a diastolic pressure based, at least in part, on the deflation of the blood pressure cuff.
